(19) United States
(12) Patent Application Publication
(10) Pub. No.: US 2025/0275912 A1
(43) Pub. Date: Sep. 4, 2025

(54) NOVEL COMPOSITION FOR IMPROVEMENT OF HAIR AND SCALP CONDITION

(71) Applicant: LUCAS MEYER COSMETICS, MASSY (FR)

(72) Inventors: ROMAIN DUROUX, COUZIEX (FR); JOAN ATTIA, LAVERNOSE LACASSE (FR)

(21) Appl. No.: 18/858,779

(22) PCT Filed: Apr. 21, 2023
(86) PCT No.: PCT/US2023/019350

§ 371 Date: Oct. 22, 2024

(30) Foreign Application Priority Data
Apr. 22, 2022 (EP) 22305603.7

(51) Int. Cl. A61K 8/9789; A61Q 5/00

(52) U.S. Cl. CPC A61K 8/9789; A61Q 5/006; A61Q 5/008

(57) ABSTRACT

An anti-inflammatory and inflammation pro-resolving composition comprising an Anetholea anisita extract for improving hair and scalp condition is provided.

### STATUS OF RELATED APPLICATION

This application claims priority to European Patent Application No. 22305603.7, filed Apr. 22, 2022, the contents hereby incorporated by reference as if set forth in its entirety.

### BACKGROUND OF INVENTION

Unhealthy scalp shares some common symptoms such as dandruff, itching, irritation, bumps on the scalp, oily hair and/or scalp, dull and unhealthy hair, or a combination thereof. Pityriasis capitis commonly known as dandruff is the most widespread and under-diagnosed scalp disorder worldwide. A healthy stratum corneum of the scalp forms a protective barrier to protect against external insults. However, a weakened stratum corneum may facilitate dandruff generation and dull and greasy hair. Evidence of a disrupted stratum corneum includes subclinical inflammation and higher susceptibility to topical irritants. Thus, targeting the inflammation in stratum corneum may improve hair and scalp condition such as dandruff.

Synthetic anti-inflammatory drugs used in anti-dandruff treatment are generally aggressive and may associate with considerable side effects. For example, ketoconazole may cause additional skin inflammation, itching and skin irritation. Another commonly used drug pyrithione zinc can cause tinging and/or burning of scalp as well as skin peeling.

Further, dandruff is a condition that requires long-term and regular treatment. It is therefore particularly important to minimize risks such as systemic adverse effects.

In responding to these needs, great effort has been made in the industry to explore and develop novel treatment solutions that are plant-based, which are generally less aggressive and safer than synthetic medicines, for preventing, treating or reducing inflammation and improving hair and scalp condition.

### SUMMARY OF THE INVENTION

The present invention provides a novel composition that prevents, treats or reduces inflammation, provides inflammation pro-resolution and tissue regeneration, and a method of use thereof. More specifically, the present invention is directed to an anti-inflammatory and inflammation pro-resolving composition comprising an Anetholea anisita extract, and its unexpected cell type-selective properties and advantageous use in improving hair and scalp condition.

In one embodiment, the present invention is directed to a composition comprising an effect amount of an Anetholea anisita extract, wherein the composition prevents, treats or reduces inflammation, provides inflammation pro-resolution and tissue regeneration, and improves hair and scalp condition.

In another embodiment, the present invention is directed to a composition comprising an effect amount of an Anetholea anisita extract, wherein the composition prevents, treats or reduces inflammation in a human hair follicle dermal papilla cell or a normal human dermal fibroblast cell.

In another embodiment, the present invention is directed to a composition comprising an effect amount of an Anetholea anisita extract, wherein the composition prevents, treats or reduces inflammation in a human hair follicle dermal papilla cell.

In another embodiment, the present invention is directed to a method of preventing, treating or reducing scalp or skin inflammation, providing inflammation pro-resolution and tissue regeneration on scalp or skin, or improving hair and scalp condition comprising the step of applying an effective amount of Anetholea anisita extract-containing composition to the scalp or skin of a subject in need thereof.

In another embodiment, the present invention is directed to a method of preventing, treating or reducing scalp inflammation, providing inflammation pro-resolution and tissue regeneration on scalp, or improving hair and scalp condition comprising the step of applying an effective amount of Anetholea anisita extract-containing composition to the scalp of a subject in need thereof.

In another embodiment, the present invention is directed to a method of improving hair or scalp condition comprising the step of applying an effective amount of Anetholea anisita extract-containing composition to the scalp of a subject in need thereof.

In another embodiment, the present invention is directed to a consumer product containing an effective amount of an Anetholea anisita extract, wherein the Anetholea anisita extract provides anti-inflammatory and inflammation pro-resolving properties.

In another embodiment, the present invention is directed to a hair and scalp care product or a skin care product containing an effective amount of an Anetholea anisita extract, wherein the Anetholea anisita extract provides anti-inflammatory and inflammation pro-resolving properties.

In another embodiment, the present invention is directed to a hair and scalp care product containing an effective amount of an Anetholea anisita extract, wherein the Anetholea anisita extract provides anti-inflammatory and inflammation pro-resolving properties.

These and other embodiments of the present invention will be apparent by reading the following specification.

### DETAILED DESCRIPTION OF THE INVENTION

Anetholea anisita is a native plant to Australia and belongs to the family of Myrtaceae. It has been produced commercially and incorporated in Australian cuisine for its flavor and aroma. Some compounds of Anetholea anisita may also possess health-enhancing properties such as antioxidant, estrogenic and/or anti-estrogenic activities, anti-inflammatory, antimicrobial and prebiotic effects. Yet, such properties may vary with different cellular targets and therefore the effect differs among different types of cells and tissues. For example, in macrophages, Anetholea anisita extract has been shown to provide a dose-dependent inhibition of the protein expression of COX-2 and, but to a less degree, of iNOS (Guo et al., Toxicology Reports (2014), 1:385-390).

In the present invention, Anetholea anisita extract has now for the first time been found to possess advantageous anti-inflammatory effect in human follicle dermal papilla cells (HFDPC) and normal human dermal fibroblast (NHDF) cells. In particular, the anti-inflammatory effect in human follicle dermal papilla cells is surprisingly superior. Specialized pro-resolving lipid mediators (SPMs) are a set of recently identified lipid mediators in the skin that provide regression of inflammatory processes, which is the prevention of the development of chronic inflammation, and restoration of tissue homeostasis. Applicants have for the first time identified the presence of SPMs in scalp and, further, demonstrated the inflammation pro-resolving effect of the Anetholea anisita extract in scalp. The Anetholea anisita extract reinforces scalp barrier, increases hair brightness, reduces dandruff, and decreases sebaceous gland volume and sebum release. Accordingly, the present invention provides a cell type and tissue-selective anti-inflammatory and inflammation pro-resolving composition comprising an Anetholea anisita extract that possesses unexpected and advantageous properties in improving hair and scalp condition.

The term “hair and scalp condition” of the present invention is understood to mean dandruff, scalp itching, scalp irritation, bumps on the scalp, oily hair and/or scalp, dull and unhealthy hair, or a combination thereof.

The term “skin condition” of the present invention is understood to mean skin inflammation, redness, sensitive skin, irritation, itching, oily skin with or without acneic tendency, imperfection, dull skin, or a combination thereof.

In the present application, an Anetholea anisita extract refers to an extract obtained from an aerial part of the plant Anetholea anisita, fresh or dried, such as leaves, flowers, stems or a combination thereof. In one embodiment, an Anetholea anisita extract is an extract obtained from the leaves.

In another embodiment, an Anetholea anisita material is pretreated with a process such as freezing, drying, freeze-drying, grinding or a combination thereof. In another embodiment, the pretreatment of the Anetholea anisita material comprises a grinding process.

Suitable methods of extraction for providing enriched extracts are known to those skilled in the art and may include, for example, but not limited to, decoction, infusion, maceration, percolation and leaching. In one embodiment, hot extraction such as decoction is conducted in water or in a hydro-alcoholic mixture. In the present invention, decoction is understood to mean the boiling of a plant material in water for a period of time to obtain a plant extract.

Extraction can be conducted using a polar solvent such as water, an alcohol, an ester, a ketone or a mixture thereof. Alcohols include, for example, but not limited to, methanol, ethanol, n-propanol, isopropanol, and polyols such as glycerol. Esters include, for example, but not limited to, propyl acetate, isopropyl acetate, butyl acetate, sec-butyl acetate, isobutyl acetate and tert-butyl acetate. Ketones include, for example, but not limited to, acetone, cyclohexanone and methyl ethyl ketone. In one embodiment, the solvent is water, an alcohol or an aqueous alcoholic mixture such as a water/ethanol, water/methanol or water/glycerol mixture. In another embodiment, the solvent is water.

In one embodiment, the Anetholea anisita extract is an aqueous extract. At the end of the extraction, the aqueous Anetholea anisita extract is recovered by removing the solid residue using centrifugation and/or filtration. Subsequently, the aqueous extract can be further (i) formulated by addition of a pH-adjusting agent, a stabilizing agent or a cosmetically acceptable vehicle or support; (ii) concentrated; and/or (iii) dried.

The Anetholea anisita extract of the present invention contains a dry matter content of about 0.01% and greater, preferably from about 0.02% to about 50%, more preferably from about 0.05% to about 10%, and even more preferably from about 0.1% to about 5%. Unless otherwise specified, percentages (% s) are by weight.

The term “aqueous extract” is understood to mean a water-based preparation of Anetholea anisita extract according to the disclosure of the present invention.

The term “a” or “an” is understood to mean one or more.

The term “an effective amount” is understood to mean the amount of an Anetholea anisita extract used, wherein the Anetholea anisita extract prevents, treats or reduces scalp or skin inflammation, provides inflammation pro-resolution and tissue regeneration on scalp or skin, or improves hair and scalp condition. More specifically, in the present invention, an effective amount of the Anetholea anisita extract provides improvement of hair and scalp condition such as dandruff, scalp itching, scalp irritation, bumps on the scalp, oily hair and/or scalp, dull and unhealthy hair, or a combination thereof.

The effective amount may vary depending on many factors such as severity and duration of the condition. An Anetholea anisita extract treatment in any amount and for any length of time that provides any degree of the improvement of hair and scalp condition can be used.

In certain embodiments, the effective amount of an Anetholea anisita extract ranges from about 0.01 to about 20% by weight, preferably from about 0.05 to about 10% by weight and more preferably from about 0.1 to about 5% by weight.

The term “a consumer composition” is understood to mean an Anetholea anisita extract-containing composition, wherein the composition prevents, treats or reduces inflammation, provides inflammation pro-resolution and tissue regeneration on scalp or skin, or improves hair and scalp condition. The consumer composition of the present invention may contain an Anetholea anisita extract at a level of about 10 to about 50% and preferably of about 20 to about 40% by weight of the consumer composition. Additional materials can also be used in conjunction with the consumer composition to encapsulate and/or deliver the anti-inflammatory and inflammation pro-resolving effect. Some well-known materials are, for example, but not limited to, polymers, oligomers, other non-polymers such as surfactants, emulsifiers, lipids including fats, waxes and phospholipids, organic oils, mineral oils, petrolatum, natural oils, perfume fixatives, fibers, starches, sugars and solid surface materials such as zeolite and silica. Some preferred polymers include polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde), or a combination thereof.

The term “a consumer product” is understood to mean an Anetholea anisita extract-containing product, wherein the product possesses the anti-inflammatory and inflammation pro-resolving properties that prevent, treat or reduce inflammation or provides inflammation pro-resolution and tissue regeneration. The consumer product of the present invention may include, for example, but not limited to, bar soaps, liquid soaps, shower gels, foam bath, skin care water, lotions, creams, milks, serums, ointments, balms, salves, gels, cream-gels, emulsions, foams, sprays, eyeshadows, sticks, lipsticks, glosses, fam, deodorants, nourishing masks, exfoliating products, and hair or scalp care products. The hair or scalp care products of the present invention include, for example, but not limited to, a shampoo, a hair rinse, a conditioner, a hair bleach, a hair color, a hair dye and a hair styling product.

The following are provided as specific embodiments of the present invention. Other modifications of this invention will be readily apparent to those skilled in the art. Such modifications are understood to be within the scope of this invention. Materials were purchased from Aldrich Chemical Company unless noted otherwise. As used herein all percentages are weight percent unless otherwise noted, L is understood to be liter, mL is understood to be milliliter, g is understood to be gram, ng is understood to be nanogram, M is understood to be molar and μM is understood to be micromolar. IFF as used in the examples is understood to mean International Flavors & Fragrances Inc., New York, NY, USA.

### Example I: Preparation of Test Samples

An aqueous Anetholea anisita extract was prepared from Anetholea anisita leaves with decoction according to a method known in the art, for example, WO2018/073714A1. Glycerol was then added to the aqueous extract to provide a hydroalcoholic mixture with a glycerol to the aqueous extract weight ratio of 70:30.

The obtained Anetholea anisita extract comprised a dry matter content of about 1%.

### Example II: Inhibition of Interleukin-8 (IL-8) Production

IL-8 is a potent neutrophil chemoattractant. An increase of IL-8 expression is associated with inflammation process. The effect of Anetholea anisita extract (prepared in Example I) on the production of IL-8 was evaluated in human follicle dermal papilla cells and normal human dermal fibroblast cells, wherein the IL-8 production was induced by the addition of IL-1α.

Cell Culture and Treatment: Human follicle dermal papilla cells (HFDPC) (Promocell) and normal human dermal fibroblast (NHDF) cells (Promocell) were cultivated according to standard procedures. Cells were treated with IL-1α (0.002 ng/mL) and incubated in the presence of Anetholea anisita extract at different concentrations for 24 hours. Cells with no treatment (“Untreated”) or treated with IL-1αbut without the Anetholea anisita extract (“Control”) served as control samples.

Cell Viability Assay: At the end of incubation, cell viability assays were conducted using CellTiter 96® A Queous MTS reagent powder (Promega) to determine cytotoxic effects, if any, of the Anetholea anisita extract. No significant difference in cell viability between the Anetholea anisita extract-treated and the control samples was observed. Thus, Anetholea anisita extract has no cytotoxic effects on human follicle dermal papilla cells and normal human dermal fibroblast cells.

IL-8 Production: IL-8 levels were quantified using ELISA (enzyme-linked immunosorbent assay). Absorbance values were measured and the scores of IL-8 levels are reported as the percentage of absorbance values in treated samples respect to control samples. Results of average IL-8 scores are reported as follows:

| | | | Anetholea anisita extract (%)* | | |
|---|---|---|---|---|---|
| Cell | Untreated | Control | 0.1 | 0.2 | 0.5 |
| HFDPC | 0 | 100 | 24 | 4 | −1 |
| NHDF Cells | 0 | 100 | 63 | 53 | 24 |

*All treated samples (“Anetholea anisita extract”) showed significant difference (p < 0.001) when compared with the control sample (“Control”).

Anetholea anisita extract significantly lowered the IL-8 levels in both HFDPC and NHDF cells (p<0.001 in paired Student's t-test). Further, its inhibition of IL-8 production is surprisingly superior in HFDPC. Thus, the anti-inflammatory properties of Anetholea anisita extract are cell type-selective. Such a finding is unpredictable.

### Example III: Inhibition of Reactive Oxygen Species (ROS) Production

Reactive oxygen species (ROS) are produced by cells that are involved in the host-defense response. The ROS act as both a signaling molecule and a mediator of inflammation. The effect of Anetholea anisita extract (prepared in Example I) on the production of ROS was evaluated in human follicle dermal papilla cells, wherein the ROS production was induced by the addition of pyocyanin.

Cell Culture and Treatment: Human follicle dermal papilla cells were cultivated according to standard procedures. Cells were treated with Anetholea anisita extract (prepared in Example I) for 30 minutes before pyocyanin (Sigma-Aldrich Corp.) was added at 100 μM. Cells were incubated for 2 more hours. Cells without Anetholea anisita extract treatment (“Control”) served as control samples.

ROS Production: ROS production was measured with fluorescence staining followed by quantitative analysis. The scores of ROS production are expressed as the percentage of fluorescent intensity in treated samples respect to control samples. Results of average ROS production scores are reported as follows:

| | Anetholea anisita extract (%)* | | |
|---|---|---|---|
| Control | 0.1 | 0.2 | 0.5 |
| 100 | 25 | 27 | 33 |

*All treated samples (“Anetholea anisita extract”) showed significant difference (p < 0.001) when compared with the control sample (“Control”).

Anetholea anisita extract significantly decreased the ROS production in HFDPC (p<0.001 in paired Student's t-test).

### Example IV: Decrease of Sebaceous Gland Volume In Ex Vivo Studies

Sebaceous glands are in charge of sebum production. Sebum overproduction may lead to inflammatory events. In the present invention, the effect of Anetholea anisita extract on the size of sebaceous glands was studied ex vivo.

Scalp Explant and Treatment: Human scalp explant was established and maintained according to a method known to those skilled in the art, for example, WO2020/157428A1. On day 0, 2, 5 and 7, Anetholea anisita extract (2%, prepared in Example I) and a placebo (glycerin) were each applied to a respective scalp explant topically. On day 0 (“Control”) and 8, scalp explant sample from each treatment was collected and fixed in formol.

Sebaceous Gland Volume: The volume of the sebaceous glands was measured using X-ray microtomography according to a method known to those skilled in the art (Bailly et al., Sci Rep. (2018), 8(1): 14003). Results of average lobe volume per gland are reported as follows:

| | | Placebo | Anetholea anisita |
|---|---|---|---|
| Treatment | Control | (Day 8) | extract (Day 8) |
| Average lobe volume (×10⁻⁷ μm³) | 1.37 | 1.22 | 1.02 |
| Change (%) | | −8.2 | −23.5* |

*Treated samples (“Anetholea anisita extract”) showed significant difference (p < 0.01) when compared with the control sample (“Control”).

Anetholea anisita extract significantly decreased the volume/size of sebaceous glands (p<0.01 in paired Student's t-test) in ex vivo studies.

### Example V: Effect on the Production of Additional Inflammation Associated Proteins

Prostaglandin E2 (PGE2) is a potent inflammatory mediator. Leukotriene B4 (LTB4) is a leukotriene that is involved in inflammation. The in vivo effect of Anetholea anisita extract (prepared in Example I) on the levels of IL-1α, IL-8, PGE2, LTB4 and specialized pro-resolving lipid mediators (SPMs) including lipoxin B4 (LXB4), resolvin D1 (RvD1) and resolvin D2 (RvD2) were evaluated.

Preparation of Test Samples: An Anetholea anisita extract (prepared in Example I)-containing shampoo at 2% was prepared based on Applicants' proprietary formula for assessing the effectiveness of Anetholea anisita extract on the production of IL-1α, IL-8, PGE2, LTB4 and SPMs including lipoxin B4 (LXB4), resolvin D1 (RvD1) and resolvin D2 (RvD2). In control samples, a placebo of a glycerin and water mixture with a glycerin to water weight ratio of 70:30 was used to replace the Anetholea anisita extract.

Testing Procedure: Forty volunteers aged 18-40 with conditions of greasy hair and dandruff were randomly divided into two groups of twenty, the treatment group with every other day treatment of the Anetholea anisita extract-containing shampoo or the control group with every other day treatment of the placebo-containing shampoo. The treatment continued for 28 days. At day 28, sebum was collected from placebo and Anetholea anisita group.

Sebum Samples and Protein Quantification: Sebum samples were collected from a scalp that had been treated with Anetholea anisita extract or placebo for 28 days. Samples were processed and the above-described proteins were quantified using a method known to those skilled in the art, for example, Le Faouder P. et al., J Chromatogr B Analyt Technol Biomed Life Sci. (2013), 922:123-133. The scores of protein levels are each reported as the percentage in treated samples respect to control samples. Results are reported as follows:

| Protein | Level |
|---|---|
| IL-1a | 8 |
| IL-8 | 52 |
| PGE2 | 61 |
| LTB4 | 56 |
| LxB4 | 198 |
| RvD1 | 162 |
| RvD2 | 172 |

Anetholea anisita extract significantly decreased the production of pro-inflammatory proteins including IL-1α, IL-8, PGE2 and LTB4 but increased the production of SPMs including lipoxin B4 (LXB4), resolvin D1 (RvD1) and resolvin D2 (RvD2).

### Example VI: Improvement of Hair and Scalp Condition

Unhealthy scalp may exhibit various abnormal biophysical properties such as scalp pH imbalance, increment in trans-epidermal water loss (TEWL), increased sebum production, dandruff condition and loss of hair gloss. The effect of Anetholea anisita extract (prepared in Example I) on these biophysical properties of hair and scalp were evaluated.

Test samples and test procedure were the same as those described in Example V. Parameters were measured according to a method known to those skilled in the art and included (i) pH (Liu et al., J Cosmet Dermatol. (2022), 00:1-10); (ii) trans-epidermal water loss (Khosrowpour et al., J Cosmet Dermatol. (2019) 18:857-861); (iii) sebum level (Rajaiah Yogesh H. et al., J Cosmet Dermatol. (2021), 00:1-10); (iv) the presence of dandruff (Cavusoglu et al., Arch Dermatol Res. (2016) 308:631-642); and (v) hair gloss (Li et al., Sci. Reports, (2017) 7:18046). The measurements are each reported as the percentage of Day 28 treated samples respect to Day 0 samples or Day 28 placebo samples.

Results of Day 28 vs Day 0 in treated samples are reported as follows:

Anetholea anisita extract
Day

| | 0 | 28 |
|---|---|---|
| pH | 4.94 | 5.39** |
| TEWL | 100 | 83.3* |
| Sebum level | 100 | 85.5** |
| Dandruff | 100 | 51.2**** |
| Hair gloss | 100 | 129.9** |

Treated samples (“Anetholea anisita extract”) showed significant difference (*p < 0.05; **p < 0.01; ***p < 0.001; ****p < 0.0001) when compared with the control sample (“Control”).

Results of treated samples vs placebo samples at Day 28 are reported as follows:

| Treatment | Placebo | Anetholea anisita extract |
|---|---|---|
| pH | 5.22 | 5.39* |
| TEWL | 100 | 94* |
| Sebum level | 100 | 83* |
| Dandruff | 100 | 72*** |
| Hair gloss | 100 | 125**** |

Treated samples (“Anetholea anisita extract”) showed significant difference (*p < 0.05; **p < 0.01; ***p < 0.001; ****p < 0.0001) when compared with the placebo samples.

1-19. (canceled)

20. An anti-inflammatory and inflammation pro-resolving composition comprising an Anetholea anisita extract.

21. The composition of claim 20, wherein the Anetholea anisita extract is presented at a level of from about 0.01 to about 20% or from about 0.1 to about 5% by weight of the composition.

22. The composition of claim 20, wherein the Anetholea anisita extract is an aqueous extract obtained by decoction from leaves.

23. The composition of claim 20, wherein the composition prevents, treats or reduces inflammation in a cell selected from the group consisting of a human hair follicle dermal papilla cell and a normal human dermal fibroblast cell.

24. The composition of claim 23, wherein the composition decreases the production of specialized pro-resolving lipid mediators (SPMs).

25. The composition of claim 20, wherein the Anetholea anisita extract is obtained by aqueous decoction of leaves.

26. The composition of claim 20 which is in the form of a consumer product.

27. The composition of claim 26, wherein the consumer product is selected from the group consisting of a bar soap, a liquid soap, a shower gel, a foam bath, a skin care water, a lotion, a cream, a milk, a serum, an ointment, a balm, a salve, a gel, a cream-gel, an emulsion, a foam, a spray, an eyeshadow, a stick, a lipstick, a gloss, a fam, a deodorant, a nourishing mask, an exfoliating product, and a hair or scalp care product.

28. The composition of claim 27, wherein the hair or scalp care product is selected from the group consisting of a shampoo, a hair rinse, a conditioner, a hair bleach, a hair color, a hair dye and a hair styling product.

29. A method of preventing, treating or reducing inflammation, providing inflammation pro-resolution and tissue regeneration, or improving hair and scalp condition comprising the step of applying an anti-inflammatory and inflammation pro-resolving composition to a scalp or a skin of a subject in need thereof, wherein the composition comprises an Anetholea anisita extract.

30. The method of claim 29, wherein the method comprising the step of applying the composition to the scalp of a subject in need thereof.

31. The method of claim 29, wherein the Anetholea anisita extract is present at a level of from about 0.05% to about 10% by weight of the composition.

32. The method of claim 29, wherein the Anetholea anisita extract is obtained by aqueous decoction of leaves.

33. The method of claim 29, which is for preventing or treating scalp irritation, scalp dandruff and/or oily scalp.

34. The method of claim 33, wherein the application of the Anetholea anisita extract in the form of the composition on the scalp decreases the production of specialized pro-resolving lipid mediators (SPMs) in the scalp.

35. The method of claim 34, wherein the composition is a hair or scalp product.

36. The method of claim 35, wherein the composition is selected from the group consisting of a shampoo, a hair rinse, a conditioner, a hair bleach, a hair color, a hair dye and a hair styling product.

37. A method of improving hair and scalp condition comprising the step of applying an anti-inflammatory and inflammation pro-resolving composition to a scalp or a skin of a subject in need thereof, wherein the composition comprises an aqueous Anetholea anisita extract.
